(19) United States
(12) Patent Application Publication
(10) Pub. No.: US 2014/0288528 A1
(43) Pub. Date: Sep. 25, 2014

(54) SINGLE-USE NEEDLE ASSEMBLY AND METHOD

(71) Applicant: Dr. Py Institute, LLC, New Milford, CT (US)

(72) Inventor: Daniel Py, Larchmont, NY (US)

(73) Assignee: Dr. Py Institute, LLC, New Milford, CT (US)

(21) Appl. No.: 14/214,701

(22) Filed: Mar. 15, 2014

Related U.S. Application Data
(60) Provisional application No. 61/800,003, filed on Mar. 15, 2013.

(51) Int. Cl. A61M 5/32

(52) U.S. Cl. CPC A61M 5/3245
USPC 604/506; 604/263

(57) ABSTRACT

A needle assembly that has a flexible shield movable between (i) a first position, where the flexible shield is uncompressed, and (ii) a second position, where the flexible shield is compressed and an administering member located within the flexible shield, where at least one of the administering member and the flexible shield is axially movable with respect to the other of the administering member and the flexible shield in an unlocked position prior to and during usage of the needle assembly, and the at least one of the administering member and the flexible shield is no longer axially movable with respect to the other of the administering member and the flexible shield in a locked position. Prior, during and after use of the needle assembly, the needle is never exposed. After a single usage of the needle assembly, the needle is locked within the flexible shield and cannot be reused.

### CROSS-REFERENCE TO RELATED APPLICATION

This patent application claims benefit under 35 U.S.C. §119 to similarly-titled U.S. Provisional Patent Application No. 61/800,003, filed Mar. 15, 2013, which is hereby incorporated by reference in its entirety as part of the present disclosure.

### FIELD OF THE INVENTION

The present invention relates to a needle assembly for injecting a substance into a patient or opposing device. In particular, the present invention relates to a single-use needle assembly that guards against accidental pricks and re-use of a needle.

### BACKGROUND OF THE INVENTION

A typical prior art needle has a sharp tip in order to inject a substance into an opposing device, membrane or a patient. One of the drawbacks of prior art needles is that a user, such as a doctor, nurse or caretaker, may accidentally prick themselves with an exposed needle. One level of concern relates to the pain and potential physical injury associated with an accidental needle prick.

A second and much more dangerous situation arises if the needle has been previously used. For example, if a needle has already been used on an individual having a certain disease, the disease may thereafter be transmitted to the user accidentally pricked with the same needle. Often times it is very difficult to determine with certainty whether a prior art needle is used or unused once out of its original packaging. Thus, at the very least, an individual accidentally pricked with a used needle may suffer great emotional distress until test results reveal whether the individual has indeed contracted a disease from the accidental needle prick.

It is an object of the present invention to overcome one or more of the above-described drawbacks and/or disadvantages of the prior art.

### SUMMARY OF THE INVENTION

In accordance with a first aspect, a device such as a needle assembly, comprises a flexible shield movable between (i) a first position, wherein the flexible shield is uncompressed, and (ii) a second position, wherein the flexible shield is compressed; and an administering member; wherein at least one of the administering member and the flexible shield is axially movable with respect to the other of the administering member and the flexible shield in an unlocked position prior to and during usage of the needle assembly, and the at least one of the administering member and the flexible shield is no longer axially movable with respect to the other of the administering member and the flexible shield in a locked position.

In some embodiments, the device further comprises a removable cap and a removable seal. In some such embodiments, the flexible shield and the administering member are sealed within the removable cap and removable seal prior to usage of the device.

In some embodiments, the administering member is a needle. In some such embodiments, the needle defines a tip at a distal end thereof.

In some embodiments, the flexible shield includes a substantially cylindrical top portion mounted atop a substantially dome-shaped base portion defining an integral spring.

In some embodiments, the flexible shield is a bellows shaped member.

In some embodiments, the administering member is in the unlocked position prior to and during a first use of the device. In some such embodiments, the administering member is in the locked position after a single use of the device.

In some embodiments, the administering member is completely enclosed within the flexible shield in the locked position.

In some embodiments, the flexible shield defines an opening at a distal end thereof extending proximally into the flexible shield, configured to align the administering member therein and prevent deflection of a distal end of the administering member.

In accordance with another aspect, a device comprises first means for shielding a second means, movable between (i) a first position, wherein the first means is uncompressed, and (ii) a second position, wherein the first means is compressed; and second means for administering a substance; wherein at least one of the second means and the first means is axially movable with respect to the other of the second means and the first means in an unlocked position, and the at least one of the second means and the first means is no longer axially movable with respect to the other of the second means and the first means in a locked position.

In some embodiments, the first means is a flexible shield and the second means is an administering member.

In accordance with another aspect, a method comprises the steps:
(i) attaching a distal end of a syringe to a needle assembly comprising: a flexible shield movable between (i) a first position, wherein the flexible shield is uncompressed, and (ii) a second position, wherein the flexible shield is compressed; and an administering member; wherein at least one of the administering member and the flexible shield is axially movable with respect to the other of the administering member and the flexible shield in an unlocked position prior to and during usage of the needle assembly, and the at least one of the administering member and the flexible shield is no longer axially movable with respect to the other of the administering member and the flexible shield in a locked position;
(ii) placing a distal end of the flexible shield adjacent a skin surface of a patient;
(iii) depressing the syringe and thereby axially moving the administering member relative to the flexible shield toward the skin surface;
(iv) moving the flexible shield from the first position to the second position;
(v) contacting the skin surface with the administering member;
(vi) penetrating the skin surface with the administering member;
(vii) introducing a substance from the syringe through the administering member, and into the skin;
(viii) retracting the syringe and thereby withdrawing the administering member from the skin;
(ix) moving the flexible shield from the second position to the first position; an
(x) locking the administering member within the shield.

In some embodiments, the distal end of the syringe comprises a connector and an annular protuberance laterally extending therefrom, and the administering member comprises a corresponding annular recess, and the attaching step comprises abutting a distal end of the administering member against the annular protuberance of the of the syringe connector. In some such embodiments, the contacting step includes snapping the annular protuberance of the syringe connector into the annular recess of the administering member. In some such embodiments, the method further comprises the step of detaching the needle assembly from the syringe. In some such embodiments, the detaching step further comprises unsnapping the administering member from the syringe connector.

In some embodiments, the penetrating step further comprises penetrating the skin surface with the administering member without exposing the administering member.

In some embodiments, the retracting step further comprises retracting the syringe and thereby withdrawing the administering member from the skin without exposing the administering member.

One advantage of the present invention is that the administering member is never exposed from the flexible shield, before during and after injection. Accordingly, a user will not prick themselves with the needle tip. Another advantage is that the administering member is locked within the flexible shield after a single use of the needle. Accordingly, the needle cannot be reused by accident on any other individual or device.

Other objects and advantages will become more readily apparent in view of the following detailed description and the accompanying drawings.

### BRIEF DESCRIPTION OF THE DRAWINGS

FIG. 1A is a perspective view of an embodiment of a needle assembly;

FIG. 1B is a perspective view of the needle assembly of FIG. 1A, showing the removable seal being peeled away;

FIG. 1C is a perspective view of the needle assembly of FIG. 1A, with the removable seal removed;

FIG. 2A is a perspective cross-sectional view of the needle assembly of FIG. 1;

FIG. 2B is a cross-sectional view of the needle assembly of FIG. 1;

FIG. 3A is a perspective view of the needle assembly of FIG. 1 prior to attachment to a syringe;

FIG. 3B is a perspective view of the needle assembly of FIG. 1 upon attachment to a syringe;

FIG. 4A is a perspective cross-sectional view of the needle assembly of FIG. 1 attached to a syringe, prior to injection of the needle;

FIG. 4B is a cross-sectional view of the needle assembly of FIG. 1 attached to a syringe, prior to injection of the needle;

FIG. 5 is a cross-sectional view of the needle assembly of FIG. 1, when the needle assembly is first placed adjacent to a skin surface at the injection point, the flexible shield is in the first position, and the annular protuberance of the syringe connector is abutting the proximal end of the needle;

FIG. 6 is a cross-sectional view of the needle assembly of FIG. 1, when the syringe is slightly depressed placing the needle tip in first contact with the skin surface, and snapping the annular protuberance of the syringe connector into the annular recess of the recess;

FIG. 7 is a cross-sectional view of the needle assembly of FIG. 1, when the flexible shield is in the second position, and the needle has penetrated the skin surface;

FIG. 8 is a cross-sectional view of the needle assembly of FIG. 1, when the flexible shield is back in the first position, and the needle has been withdrawn from the skin and locked within the shield;

FIG. 9 is a perspective view of another embodiment of a needle assembly;

FIGS. 10A AND 10B are perspective views of the needle assembly and hood of needle assembly of FIG. 9;

FIGS. 11A and 11B are perspective views of the sliding shutter of the needle assembly of FIG. 9;

FIGS. 12A and 12B are exploded views of the needle assembly of FIG. 9 showing a first and second spring embodiments;

FIG. 13A is a partial cross-sectional view of the needle assembly of FIG. 9 including the first spring embodiment;

FIG. 13B is a partial cross-sectional view of the needle assembly of FIG. 9 including the second spring embodiment;

FIGS. 14A and 14B are partial cross-sectional views of the needle assembly of FIG. 9 showing a cam system;

FIGS. 15-16E are cross-sectional views showing the progression of the sliding shutter during actuation of the needle assembly of FIG. 9;

FIGS. 16F-16H are cross-sectional views showing the progression of the sliding shutter during retraction of the needle assembly of FIG. 9; and

FIG. 16I is a perspective view of the needle assembly of FIG. 9 in a partially depressed or retracted state.

### DETAILED DESCRIPTION OF EMBODIMENTS OF THE INVENTION

In FIG. 1A a device is indicated generally by the reference numeral 10. In the illustrated embodiment, the device 10 is a needle assembly. However, as may be recognized by those skilled in the pertinent art based on the teachings herein, the device may be embodied in and otherwise may be applicable to devices other than needle assemblies, such as, for example, cannula and probe assemblies.

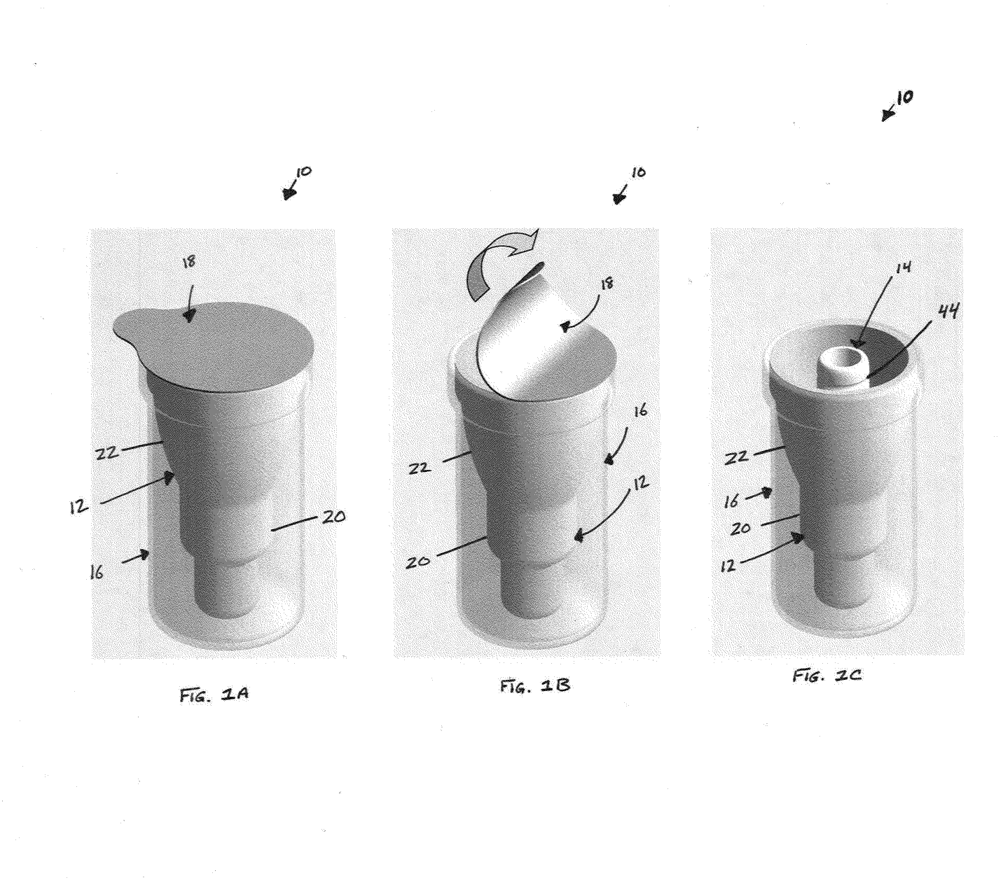

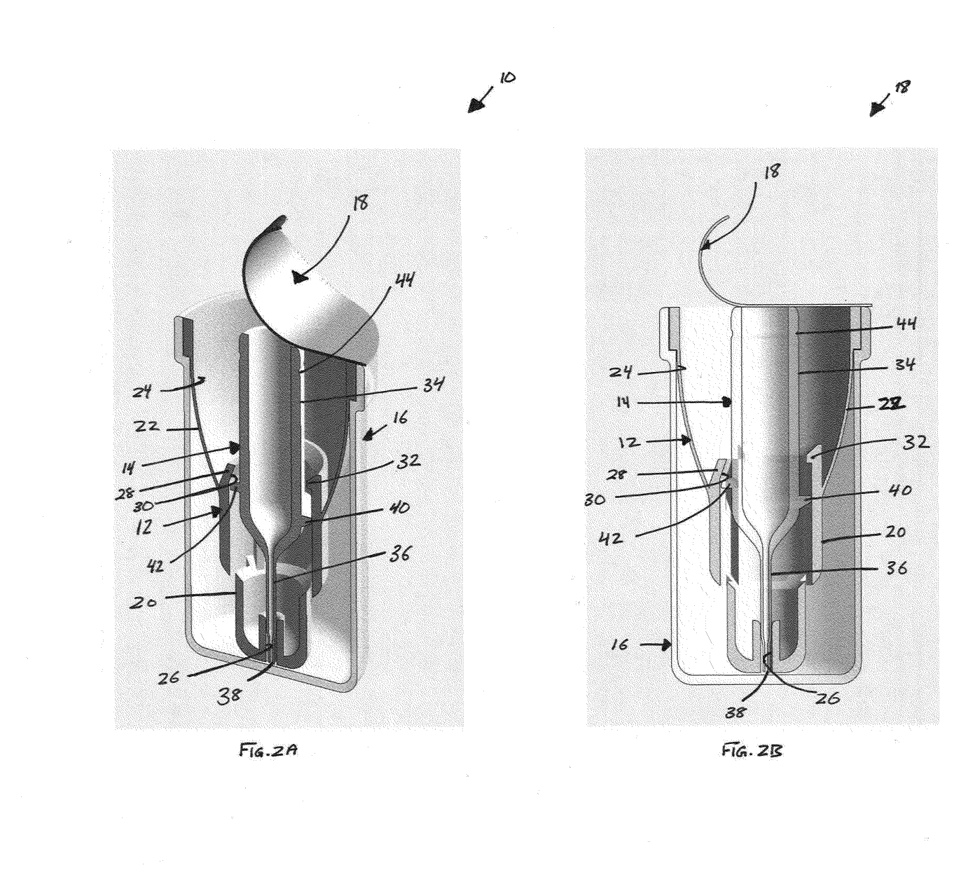

As shown in FIGS. 2A-2B, the needle assembly 10 comprises a flexible shield 12, a needle 14 centered and enclosed within the flexible shield 12, and a cap 16 removably mounted atop the flexible shield. In the illustrated embodiment, the needle assembly 10 is removably attachable to a syringe 60, such as, for example, a multiple dose syringe. An exemplary multiple dose syringe is disclosed in co-pending U.S. patent application Ser. No. 13/743,661, filed Jan. 17, 2013, entitled “Multiple Dose Syringe and Method,” which, in turn, claims the benefit of similarly title U.S. Provisional Patent Application Ser. No. 61/587,500, filed Jan. 17, 2012, which is hereby expressly incorporated by reference in its entirety as part of the present disclosure. In the illustrated embodiment, the needle assembly 10 is used for administering a substance to a patient via injection. However, as may be recognized by those of ordinary skill in the pertinent art based on the teachings herein, the needle assembly may be used for any of numerous applications currently known, or that later becomes known, such as, for example, filling a syringe with a substance, dispensing a substance from a syringe into an opposing device, and any form of fluid transfer. As also may be recognized by those of ordinary skill in the pertinent art based on the teachings herein, the needle assembly is attachable to any of numerous devices, currently known or that later become known, capable of performing the function of a syringe as described herein.

In the illustrated embodiment, the needle assembly 10 is intended for single-use. As shown in FIGS. 1A-1C, prior to usage, a removable seal 18 covers an attachment or proximal end of the cap 16, sealing the shield 12 and needle 14 therein. Therefore the needle assembly 10 is maintained in a sterile state prior to use. When ready to attach the needle assembly 10 to a syringe 60, the seal 18 is removed or peeled away from the proximal end of the cap. In the illustrated embodiment, the seal is a foil seal. However, as may be recognized by those of ordinary skill in the pertinent art based on the teachings herein, the removable seal may take the form of any of numerous different sealing mechanisms that are currently known, or that later becomes known, such as, for example, a removable liner or a removable cap.

As shown in FIGS. 2A-2B, the flexible shield 12 comprises a substantially cylindrical top portion 20, atop a substantially dome-shaped base portion 22, defining an integral spring 24. The flexible shield 12 is formed of a resilient and/or elastomeric material. The approximately dome-shaped portion allows the flexible shield 12 to move between a first position, as shown in FIG. 5, where the dome-shaped portion 22 is uncompressed, and a second position, as shown in FIG. 7, where the dome-shaped portion 22 is compressed, as described further below. The integral spring 24 naturally biases the flexible shield 12 from the compressed second position to the uncompressed first position. As may be recognized by those of ordinary skill in the pertinent art based on the teachings herein, the flexible shield may be configured in any of numerous configurations currently known, or that later become known, capable of performing the function of the flexible shield as described herein, such as, for example, a bellows configuration.

The substantially cylindrical top portion 20 defines a centered opening or guide 26 at a distal end thereof, and proximally extending into the top portion, as shown in FIGS. 2A-2B. The opening 26 receives a distal end of the needle 14 to align and guide the needle tip 38 and prevent deflection thereof, as described further below. At the junction of the top portion 20 and the base portion 22 of the flexible shield 12, the top portion defines an annular tapered protuberance 28. As can be seen, the tapered protuberance 28 defines a tapered surface 30 on the needle side thereof allowing a corresponding second projection 42 of the needle 14 to slide and snap over the surface 30 of the protuberance 28 when the needle slides in a direction from the distal end of the shield 12 toward the proximal end thereof, and thereafter preventing the needle from sliding back in the opposite direction, as described further below. The top portion 20 further defines an annular inwardly extending lateral stop surface 32, at the proximal end thereof, proximally adjacent to the tapered protuberance 28. The stop surface 32 engages and stops a first projection 40 of the needle 14 from further axial movement in the direction from the distal end of the shield 12 toward the proximal end thereof once the needle snaps over the tapered protuberance 28, as described further below. Consequently, once the needle 14 snaps over the tapered protuberance 28, the needle is locked within the shield 12.

As shown in FIGS. 2A-2B, the needle 14 includes a proximal hollow shaft 34 tapering into a relatively thinner distal hollow shaft 36. A distal end of the distal shaft 36 defines the needle tip 38. An exemplary needle is disclosed in co-pending U.S. Provisional Patent Application Ser. No. 61/635,258, filed Apr. 18, 2012, entitled “Self Closing Connector,” and similarly-titled U.S. Provisional Patent Application Ser. No. 61/625,663, filed Apr. 17, 2012, both of which are hereby expressly incorporated by reference in their entireties as part of the present disclosure. The needle may be metal, plastic, such as Graphene or Vectra, or may be made of a flexible polymer, such as a biocompatible polymer. The distal end of the distal shaft 36 frictionally fits within the centered opening 26, thereby preventing axial movement of the needle in the absence of a force overcoming the frictional force. However, as may be recognized by those of ordinary skill in the pertinent art based on the teachings herein, the needle shaft may fit within the centered opening in any of numerous configurations currently known, or that later becomes known, capable of performing the function of the frictional fit as described herein.

As can be seen, the proximal shaft 34 defines a first projection 40, extending laterally outwardly from the proximal shaft 34 adjacent the tapering distal end thereof. The first projection 40 is engageable with the stop surface 32 of the shield 12, in order to prevent axial sliding of the needle past the stop surface in the direction from the distal end of the shield 12 toward the proximal end thereof. The proximal shaft further defines a second projection 42, extending laterally outwardly from the proximal shaft 34, proximally adjacent to the first projection 40. The second projection 42 is slideable over the surface 30 of tapered protuberance 28, allowing the needle to snap over the protuberance 28 in the direction from the distal end of the shield 12 toward the proximal end thereof, and thereafter prevents the needle from sliding in the opposite direction thereafter, as explained above. The open proximal end of the proximal shaft 34 is engageable with a connector 62 of the syringe 60, to place the needle 14 in fluid communication with the syringe 60. The proximal shaft 34 defines an annular recess 44 in the sidewall thereof, distally adjacent to the proximal end of the shaft 34, engageable with a corresponding annular protuberance 64 of the connector 62, as described further below.

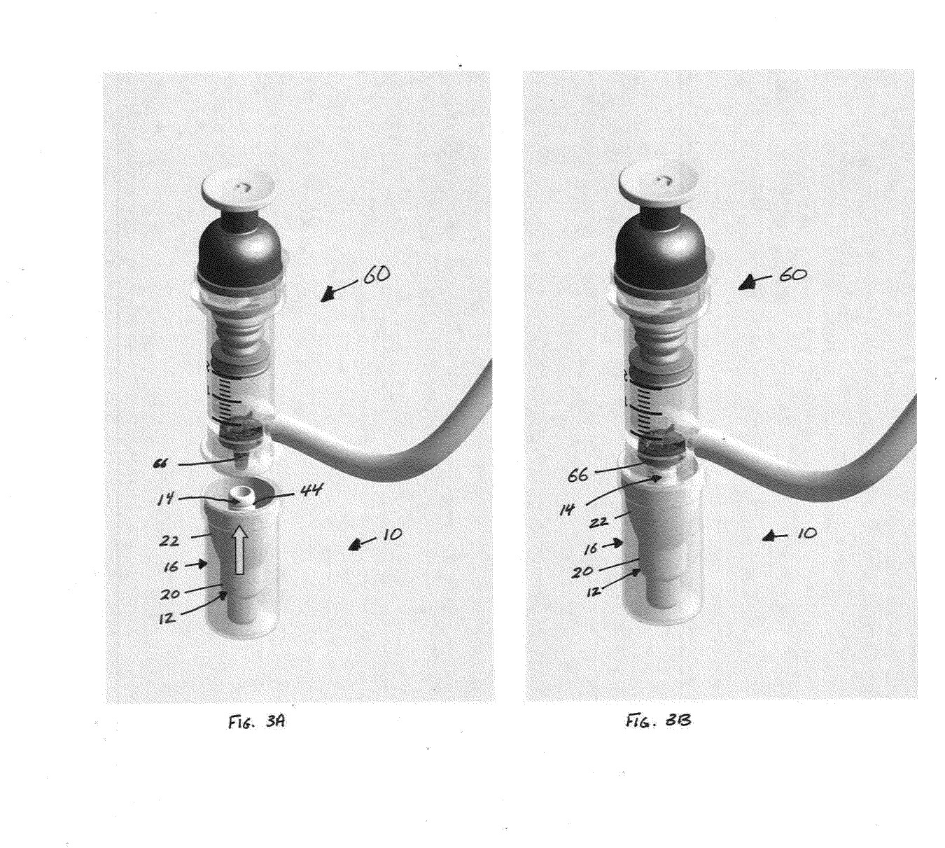

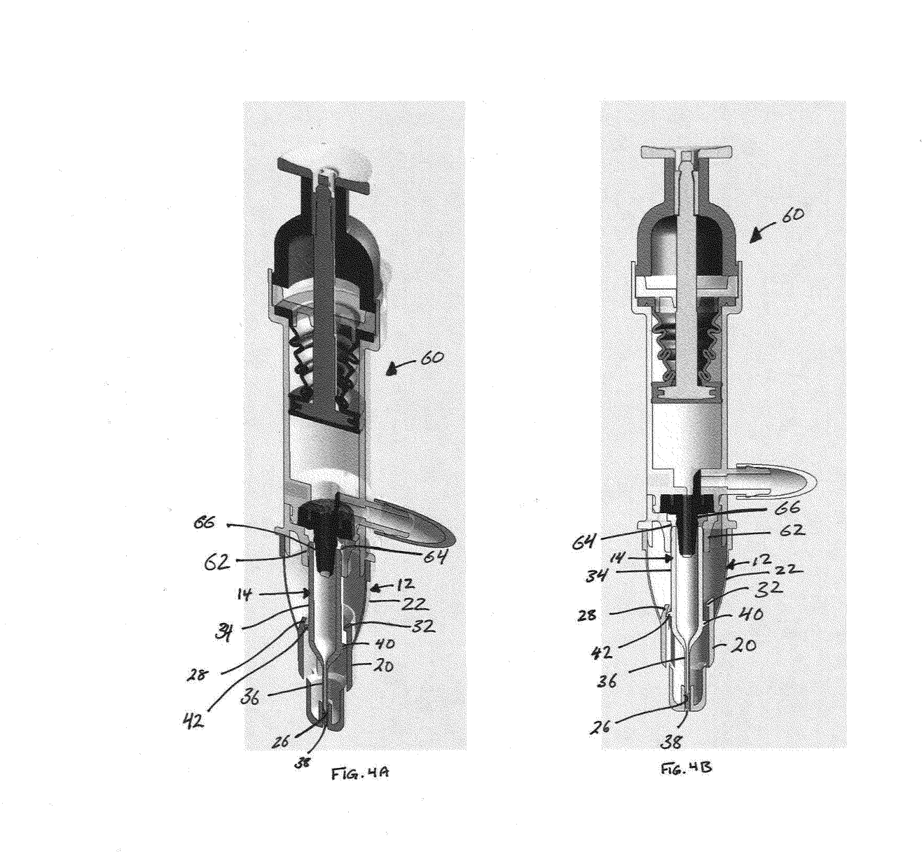

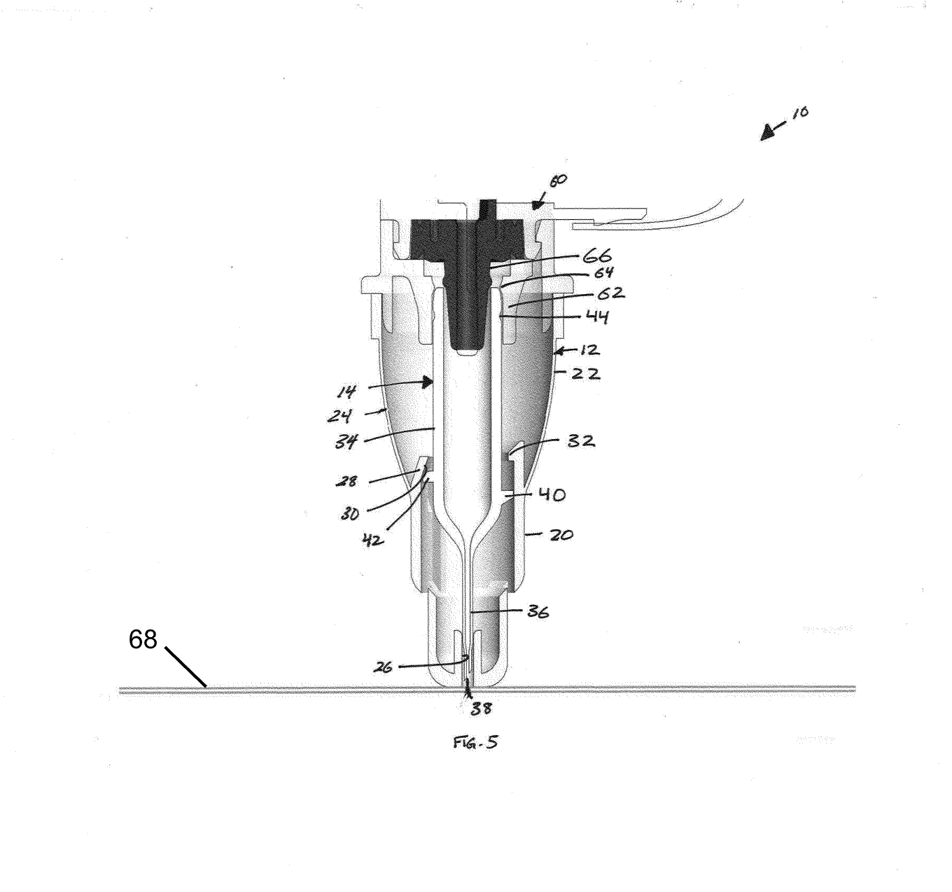

When ready for use, the seal 18 is removed from the attachment end of the cap 16, and the needle assembly 10 is attached to the syringe 60, as shown in FIGS. 3A-3B. The needle 14 is completely enclosed by the flexible shield 12 prior to and upon attachment to the syringe. In the illustrated embodiment, the proximal end of the flexible shield 12 frictionally fits about the distal end of the syringe 60. However, as may be recognized by those of ordinary skill in the pertinent art based on the teachings herein, the needle assembly may be attachable to the syringe in any of numerous different ways, currently known or that later become known, such as, for example, a luer attachment, a snap on/off attachment, and a détente attachment. When first attached, the proximal end of the needle only abuts the annular protuberance 64 of the syringe connector 62, as shown in FIGS. 4A-4B and FIG. 5. In this position, the needle 14 prevents the syringe dispensing valve 66, located within the connector 62 from opening. Thus, in this position, the syringe 60 is prevented from dispensing substance.

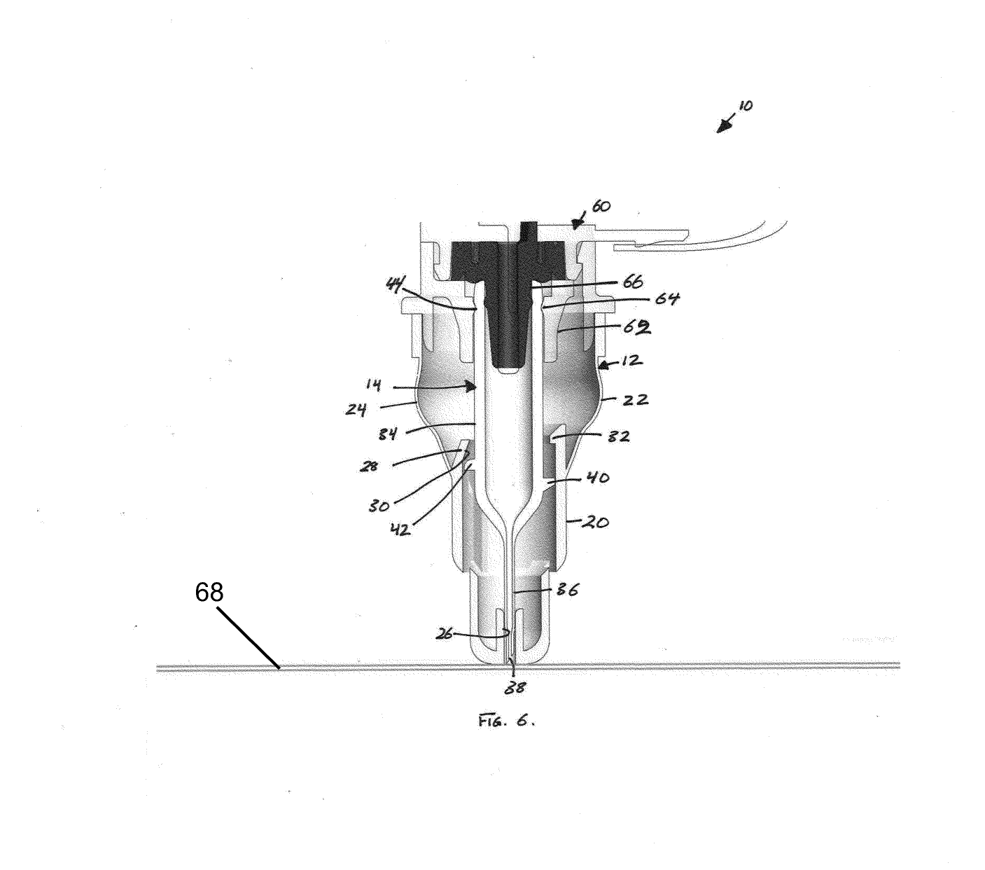

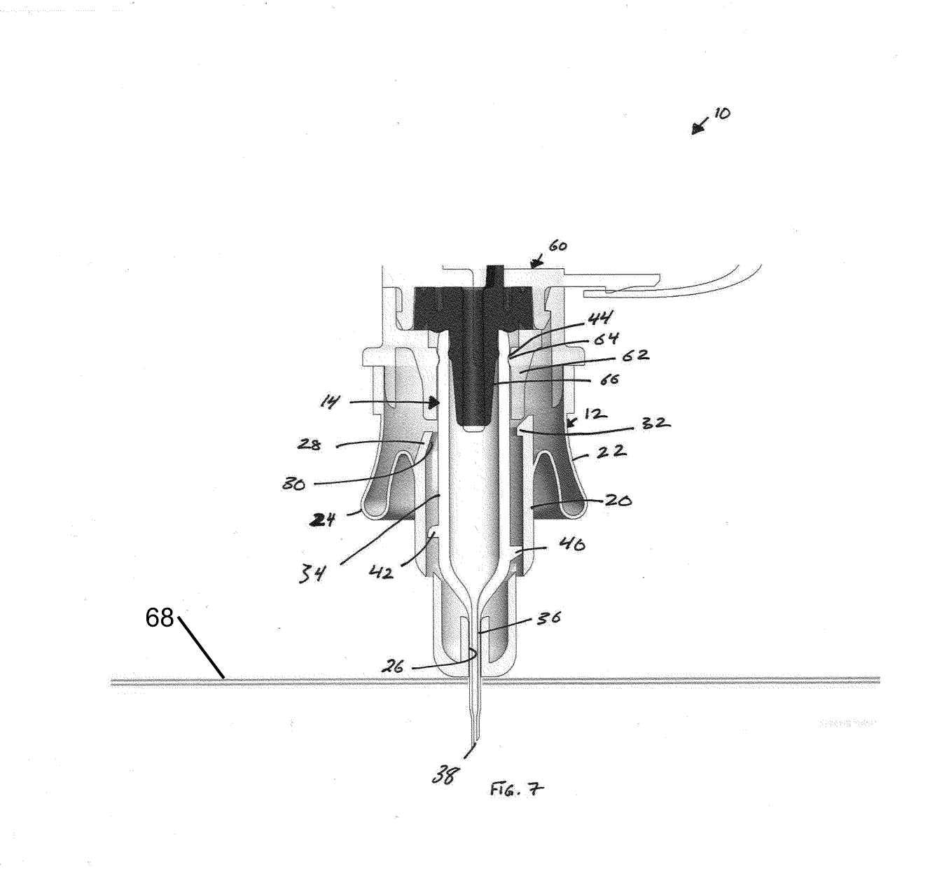

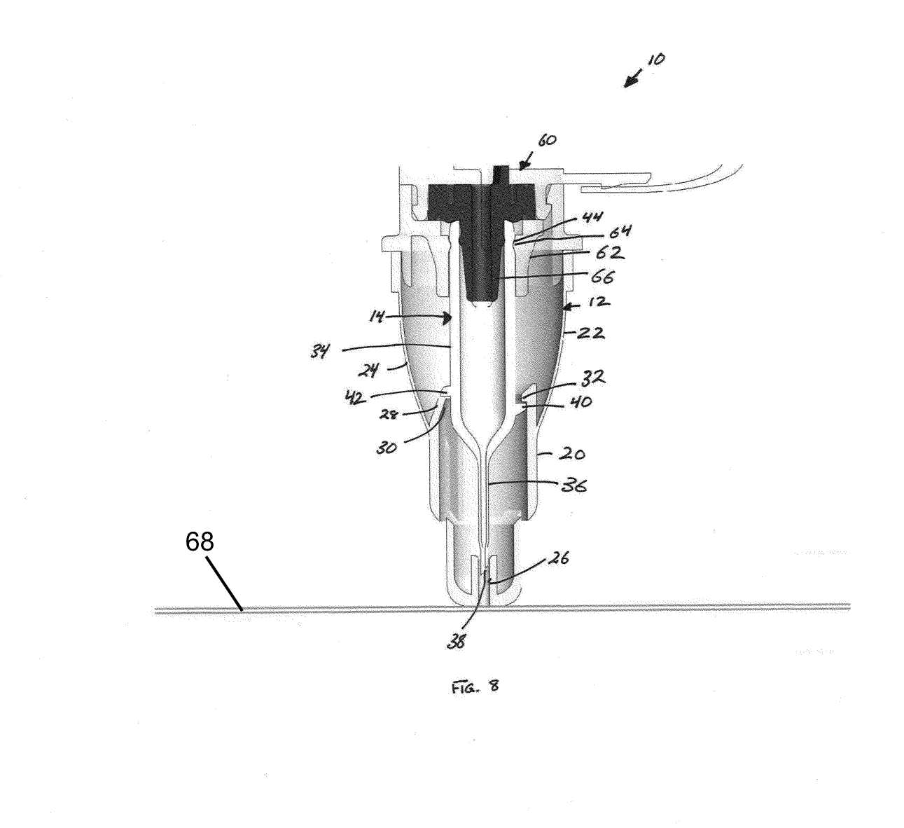

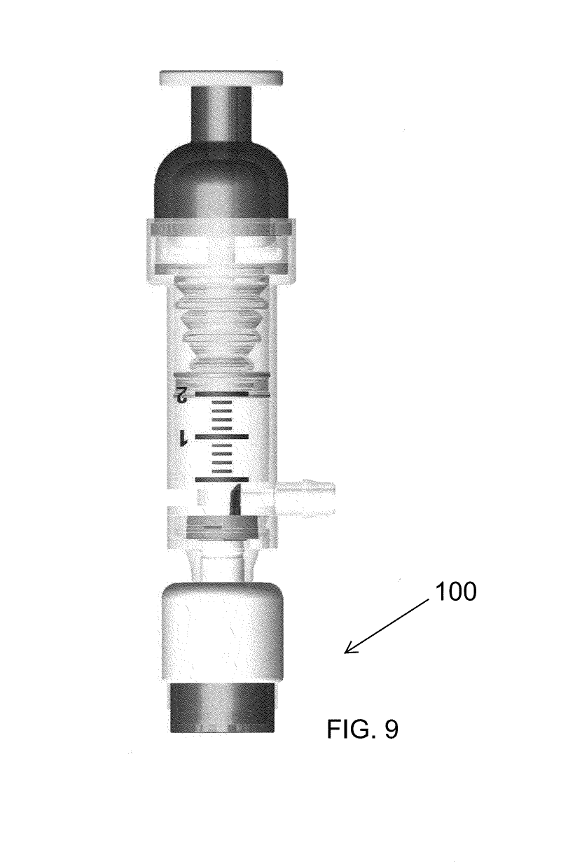

As shown in FIG. 4A-4B, the second projection 42 of the needle 14 is not yet snapped past the tapered protuberance 28 of the shield 12 when needle assembly is first attached and prior to use. The needle assembly cap 16 is then removed and the distal end of the shield 12 is placed adjacent to the skin surface at the point of injection, as shown in FIG. 5. The skin surface behaves as a stop surface for the shield 12. The syringe 60 is then depressed, as shown in FIG. 6, progressively inducing the flexible shield 16 from the first uncompressed position toward the second compressed position. As the syringe is depressed, the depression force overcomes the frictional force between the needle 14 and the opening 26 of the shield, and the needle is also depressed through the opening 26 toward the skin surface. Upon initial contact of the needle tip 38 with the skin surface, the resistance of the skin surface to puncture induces the syringe connector 62 to fully engage the proximal end of the needle 14, wherein the annular protuberance 64 of the connector moves from abutting the proximal end of the needle 14 to snapping into the annular recess 44 of the needle, as shown in FIG. 6. In this position, the needle 14 no longer prevents the syringe dispensing valve 66 from opening. Thereafter, upon further depression of the syringe 60, the needle tip 38 penetrates the skin surface, as shown in FIG. 7. During injection, the shield 12, adjacent to the skin surface, covers any portion of the needle 14 not penetrating the skin 68.

After injecting the required dose of substance, the syringe 60 is retracted, thereby withdrawing the needle 14 from the skin 68 of the patient. As the syringe is withdrawn, the distal end of the shield 12 remains adjacent to the skin surface and the integral spring 24 of the shield 12 correspondingly progressively returns the flexible shield 12 from the compressed second position back to the uncompressed first position. Consequently, the needle 14 progressively returns back into the flexible shield 12, without ever being exposed to the user. Thus, the needle is never exposed before, during, and after injection.

As the flexible shield 12 fully returns from the compressed second position to the uncompressed first position, in conjunction with the needle 14 being retracted from the skin 68 in the opposing direction, the second projection 42 of the needle 14 slides and snaps over the tapered protuberance 28 of the shield 12, as shown in FIG. 8. Thereafter the needle 14 may not be reused because the tapered protuberance 28 prevents the needle 14 from moving back toward the skin surface 68.

In order to dispose of the needle assembly 10, the shield 12 is removed from the distal end of the syringe 60. Although the annular protuberance 64 of syringe connector 62 is snapped into the annular recess 44 of the needle 14, the needle cannot be separated from the shield 12 when the shield is pulled away from the syringe because the stop surface 32 of the shield engages the first projection 40 of the needle, and the needle is locked within the shield. Thus, the needle 14 is forced to unsnap from the syringe connector as the shield 12 is removed from the syringe 60. The needle 14 therefore remains fully enclosed by the shield 12 and is locked in place within the shield with the first and second protuberances 40, 42, locked between the stop surface 32 and the tapered protuberance 28 of the shield. The single-use needle assembly may then be disposed of.

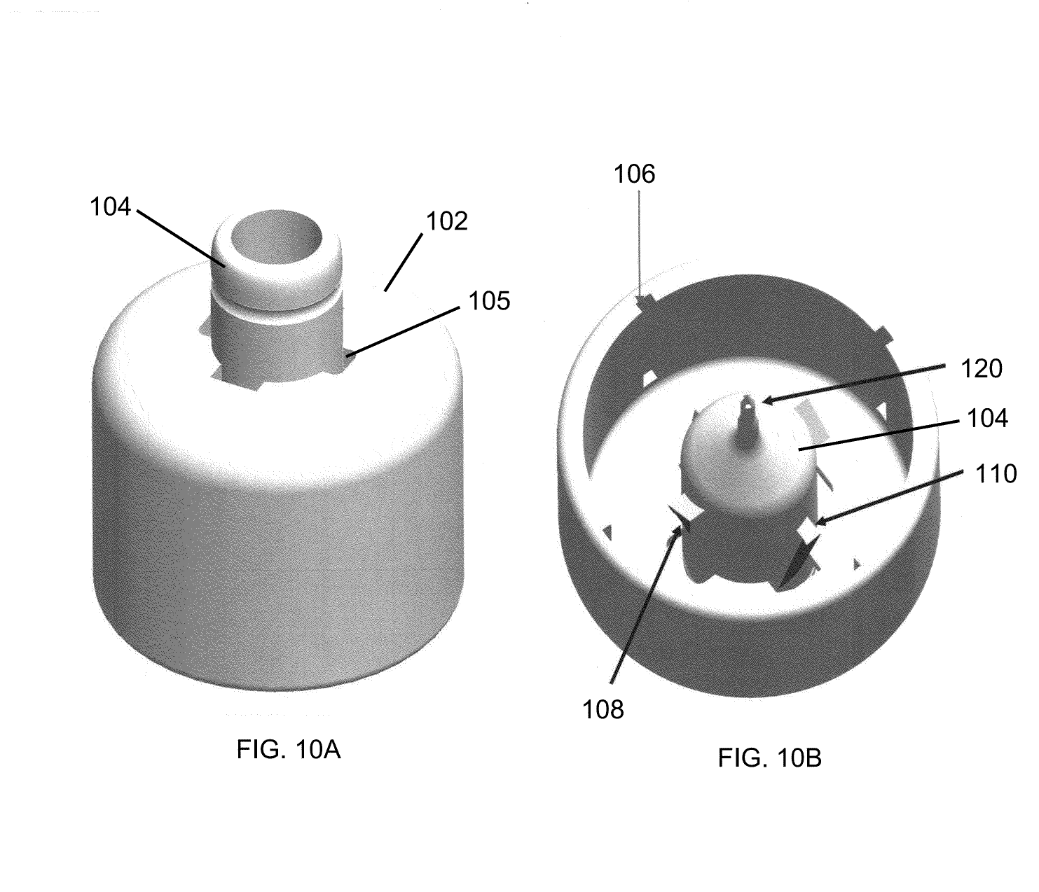

FIG. 10 is an embodiment of another device that is indicated generally by the reference numeral 100. In the illustrated embodiment, the device 100 is a needle assembly. However, as may be recognized by those skilled in the pertinent art based on the teachings herein, the device may be embodied in and otherwise may be applicable to devices other than needle assemblies, such as, for example, cannula and probe assemblies.

In the illustrated embodiment, the needle assembly 100, like the needle assembly 10, is configured for single-use. FIGS. 10A and 10B and subsequent figures illustrate a housing member 102, or hood, and a needle 104. At the upper end of the needle 104 (as oriented as in FIGS. 10A and 10B), an injection member 120 extends. The injection member 120 is configured to penetrate the skin and inject a substance under the skin therethrough. The injection member 120 can be a non-coring needle, or any other known piercing member that is capable of piercing the skin and dispersing a substance therefrom.

As can be seen in FIG. 10A, the housing member 102 includes an opening 105 therein through which a needle 104 can extend. As shown in FIG. 10B, the housing member 102 includes a plurality of slotted guides 106. The slotted guides 106 are part of a key-slot locking system, further described below, to substantially prevent relative rotation of the parts during operation of the device 100. The slots 106 can be any of a variety of shapes that are commonly known or should become known and orientated at a variety of different angles, including, in the illustrated embodiment for example, in the radial direction, i.e., 90 degrees from wall of the housing.

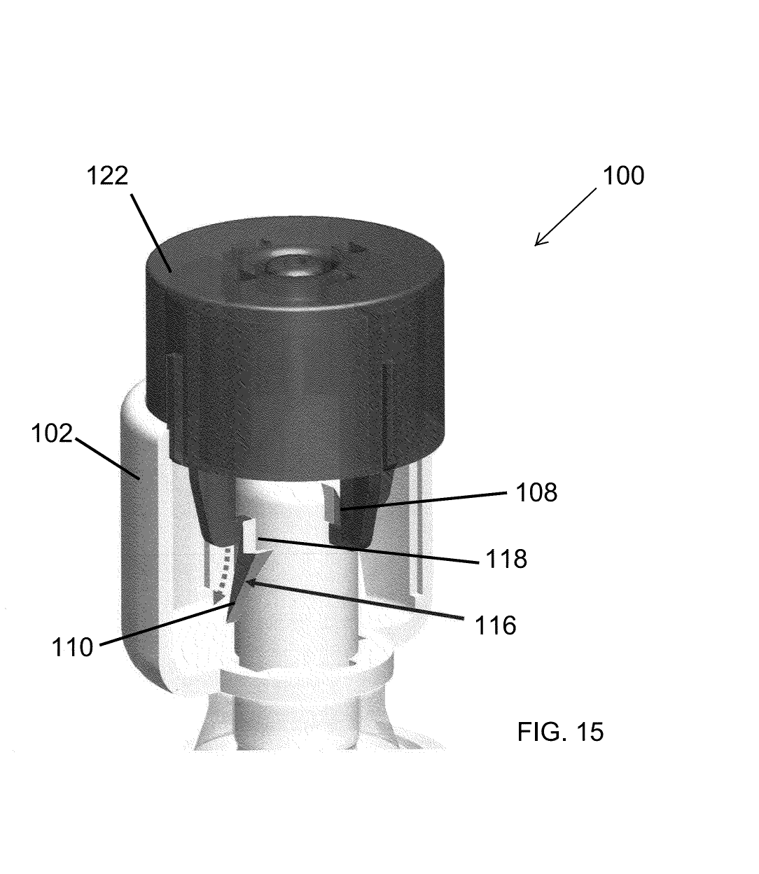

The needle 104 includes thereon a plurality of snapping cams 108 and a plurality of anti-second use cams 110 protruding therefrom and an injection member 120 extending therefrom. The snapping cams 108 and the anti-second use cams 110 are alternatively arranged about the periphery of the needle 104. In FIG. 15, the snapping cams 108 are orientated 180 degrees from each other about the periphery of the needle 104 and the anti-second use cams 110 are arranged 180 degrees from each other about the periphery of the needle 104. As illustrated, the snapping cams 108 and anti-second use cams 110 are offset from each other 90 degrees about the periphery of the needle 104. However, in other embodiments, the snapping cams 108 and anti-second use cams 110 can be offset from each other at other angles than 90 degrees, or not offset at all. In addition, though the illustrated embodiment has two snapping cams 108 and two anti-second use cams 110, the needle 104 can have fewer or more than two snapping cams 108 and two anti-second use cams 110.

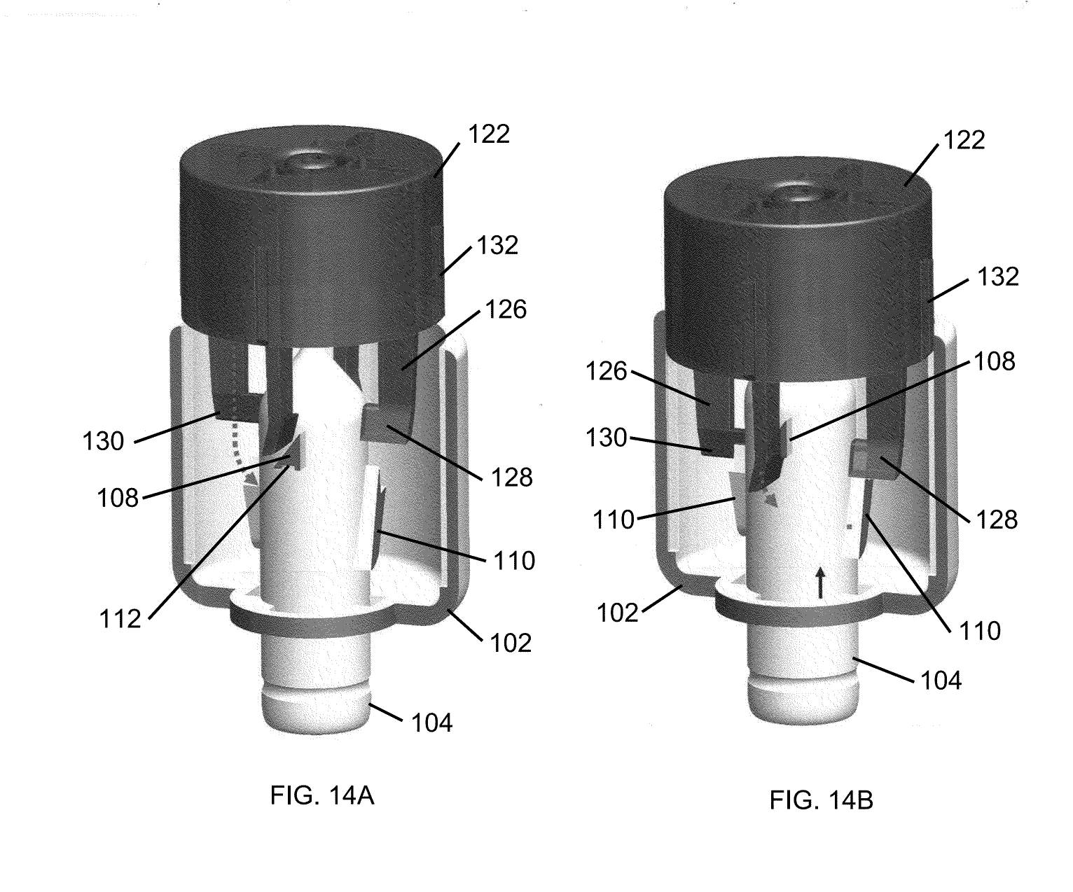

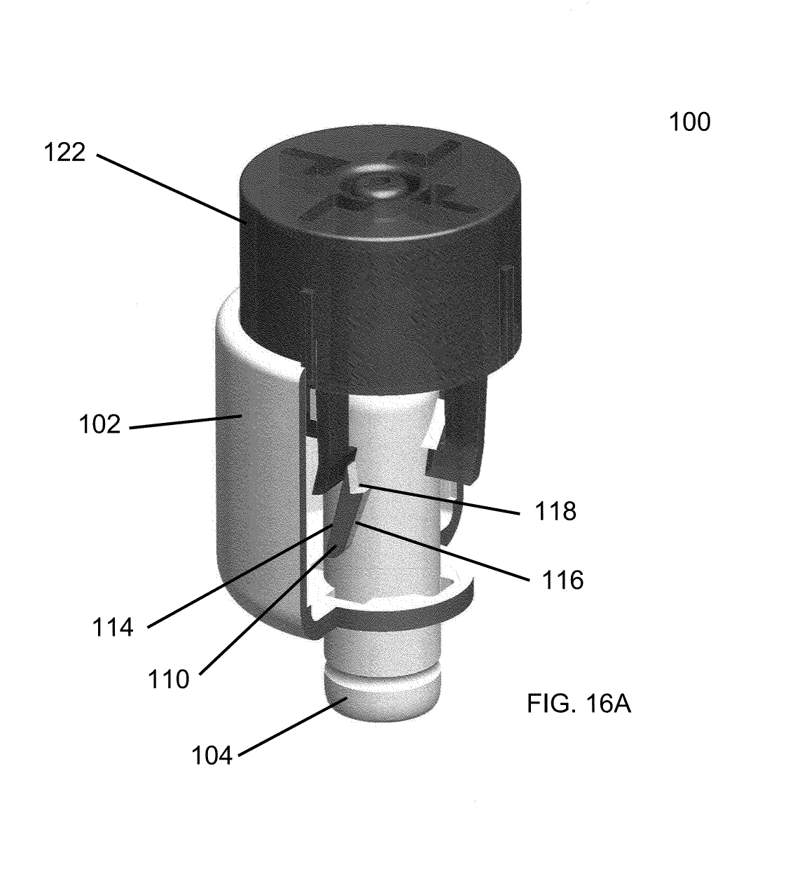

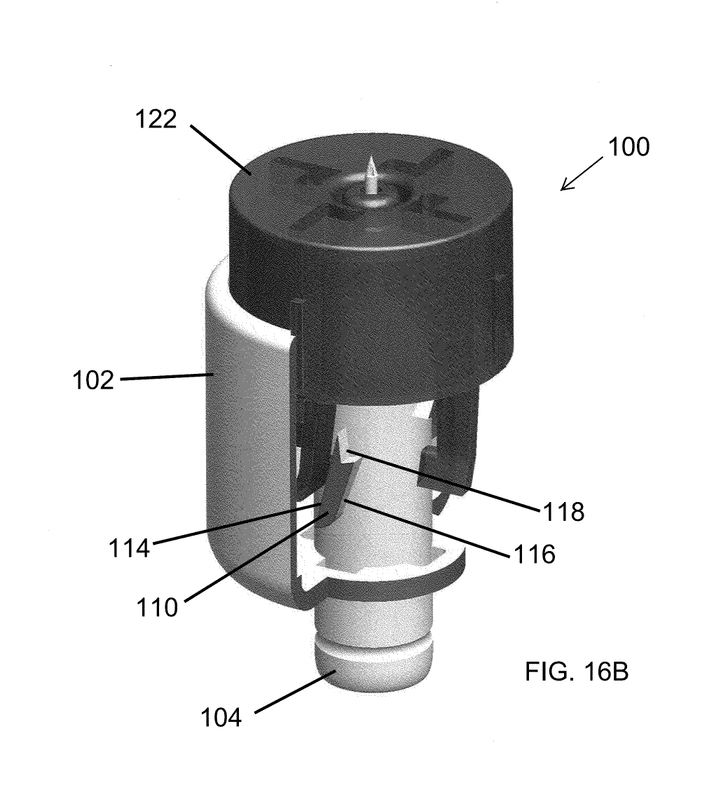

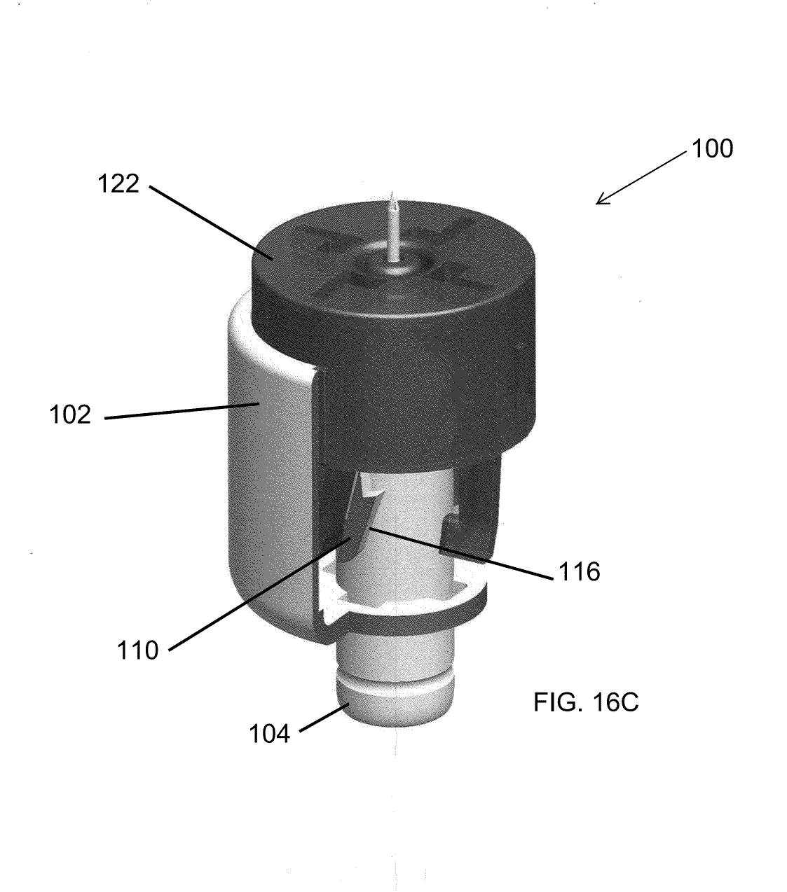

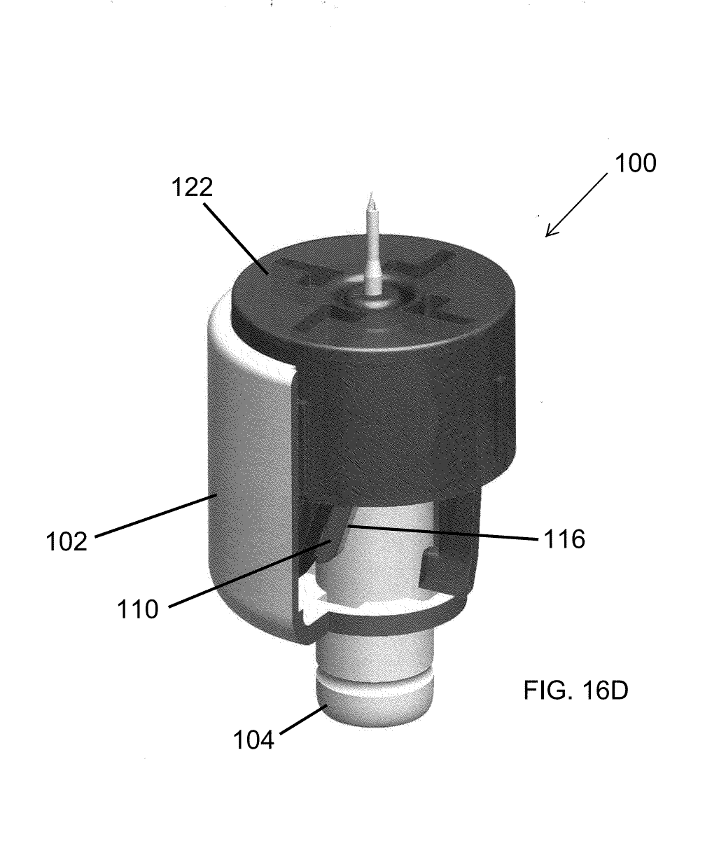

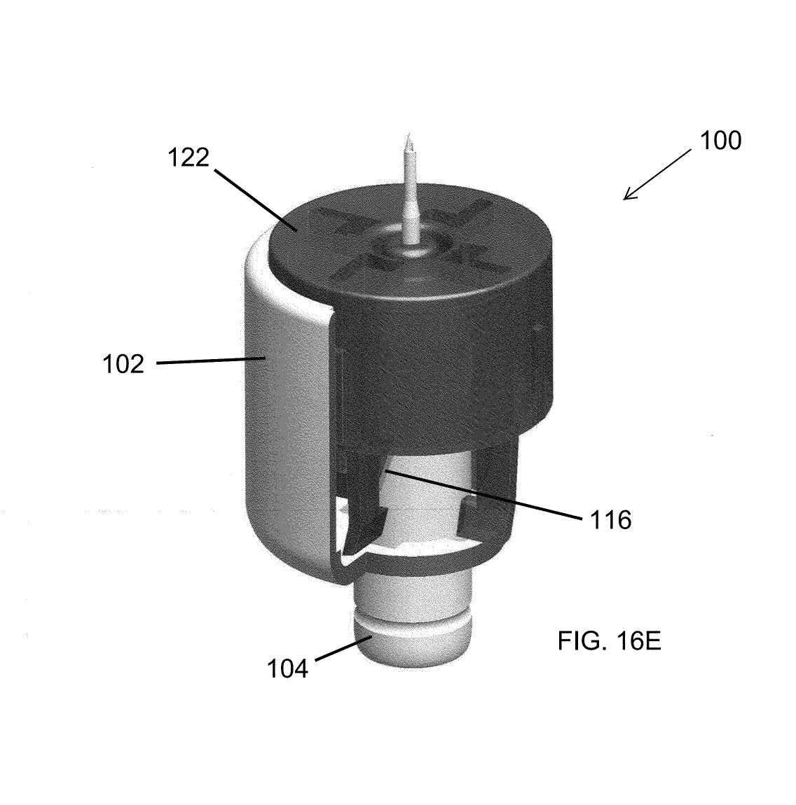

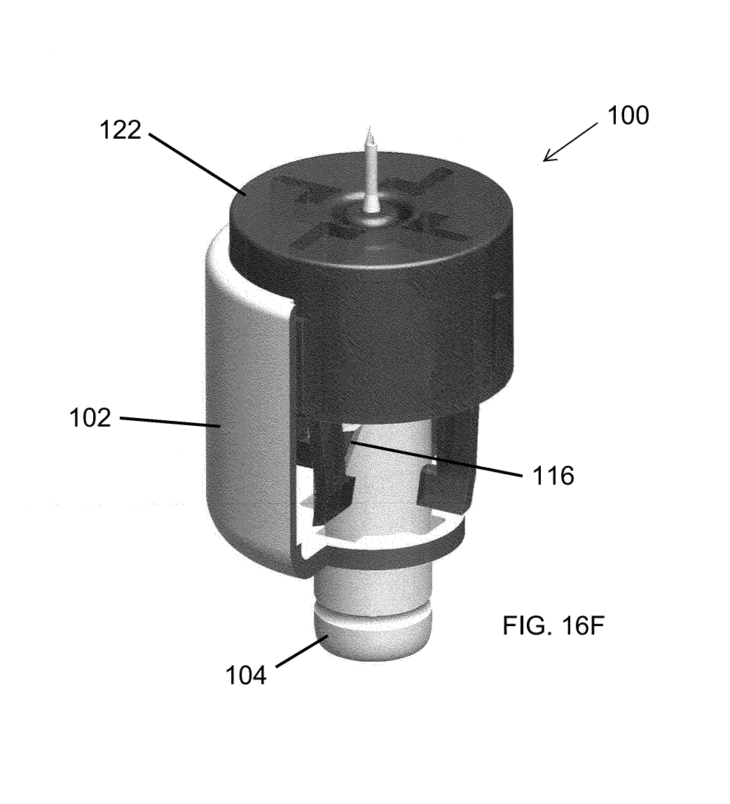

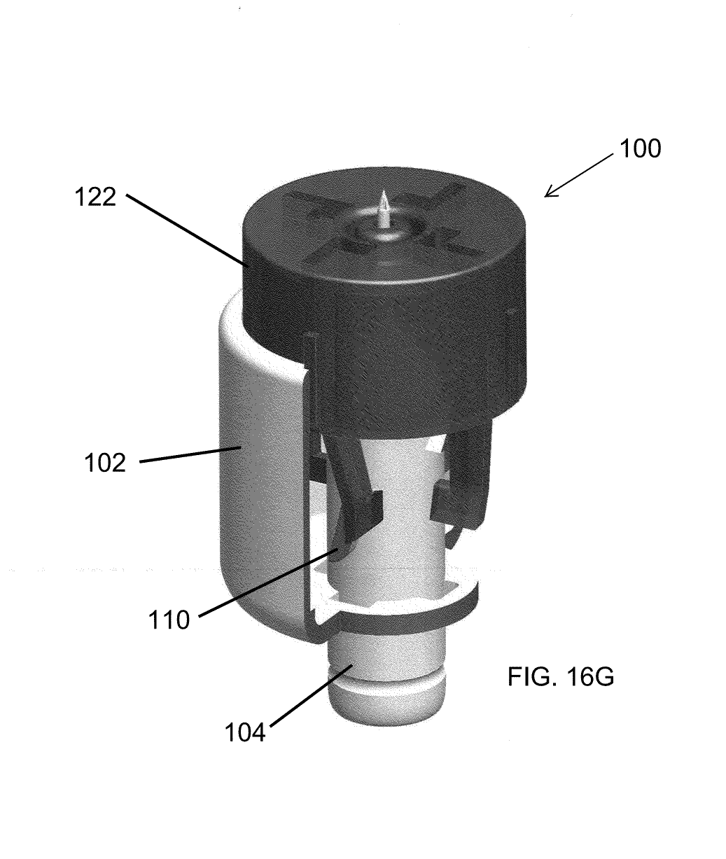

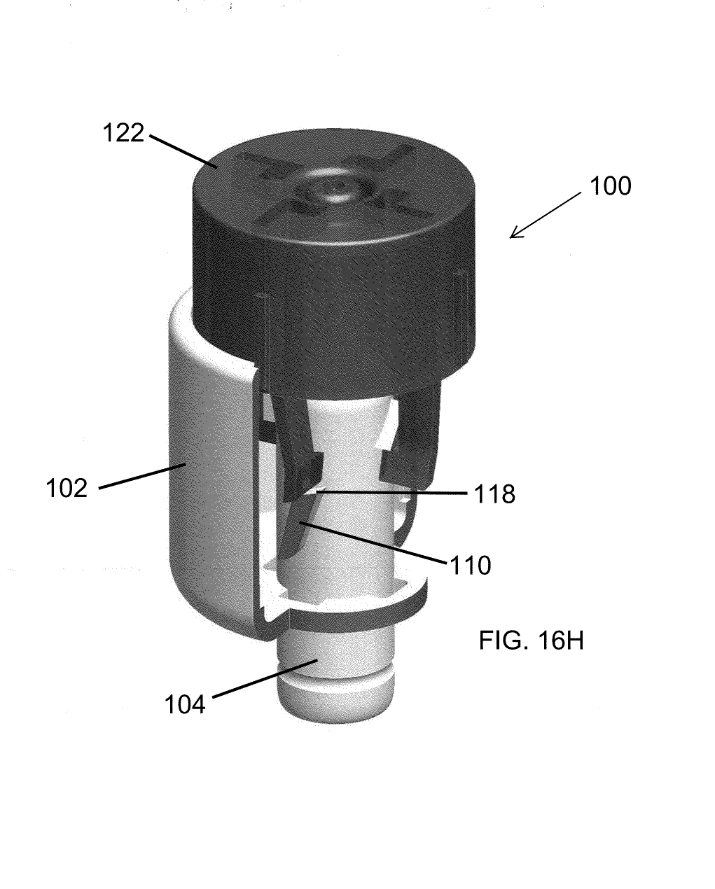

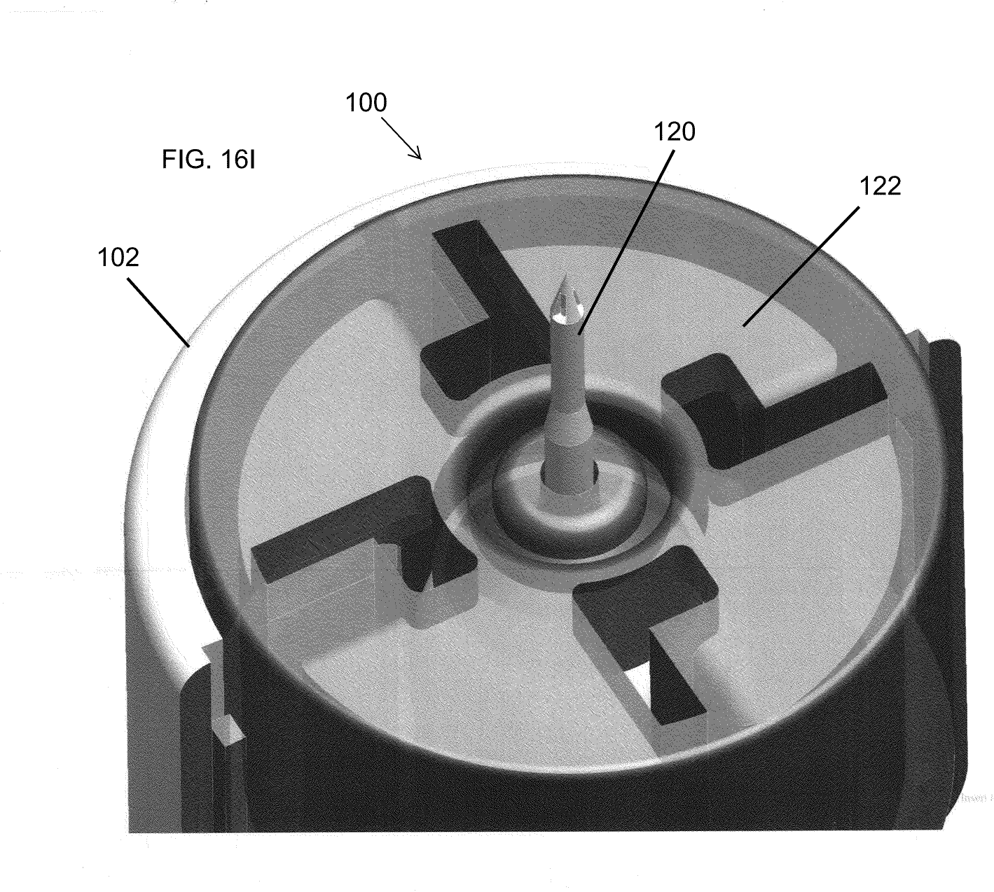

As shown in FIG. 14A, for example, the snapping cams 108 are substantially triangular shaped and include a recess 112 formed on a lower surface (as oriented in FIG. 14A) therein. The anti-second use cams 110, as shown in FIG. 16A for example, include at least a substantially sloping first side surface 114 extending downwardly (as oriented in FIG. 16A a substantially sloping second side surface 116 extending upwardly from the lower end of the first side surface 114, at an angle that, at least at a lower portion of the second side surface 116, is different from the first surface 114, and a stop region 118 defining a recess formed between the first surface 114 and the second surface 116 at the upper end of the anti-second use cam 110. Though in the illustrated embodiment the snapping cams 108 and anti-second use cams 110 have the configuration shown, in other embodiments, the snapping cams 108 and anti-second use cams 110 can take the form of any suitable shape and configuration that performs the same or substantially the same function and/or purpose.

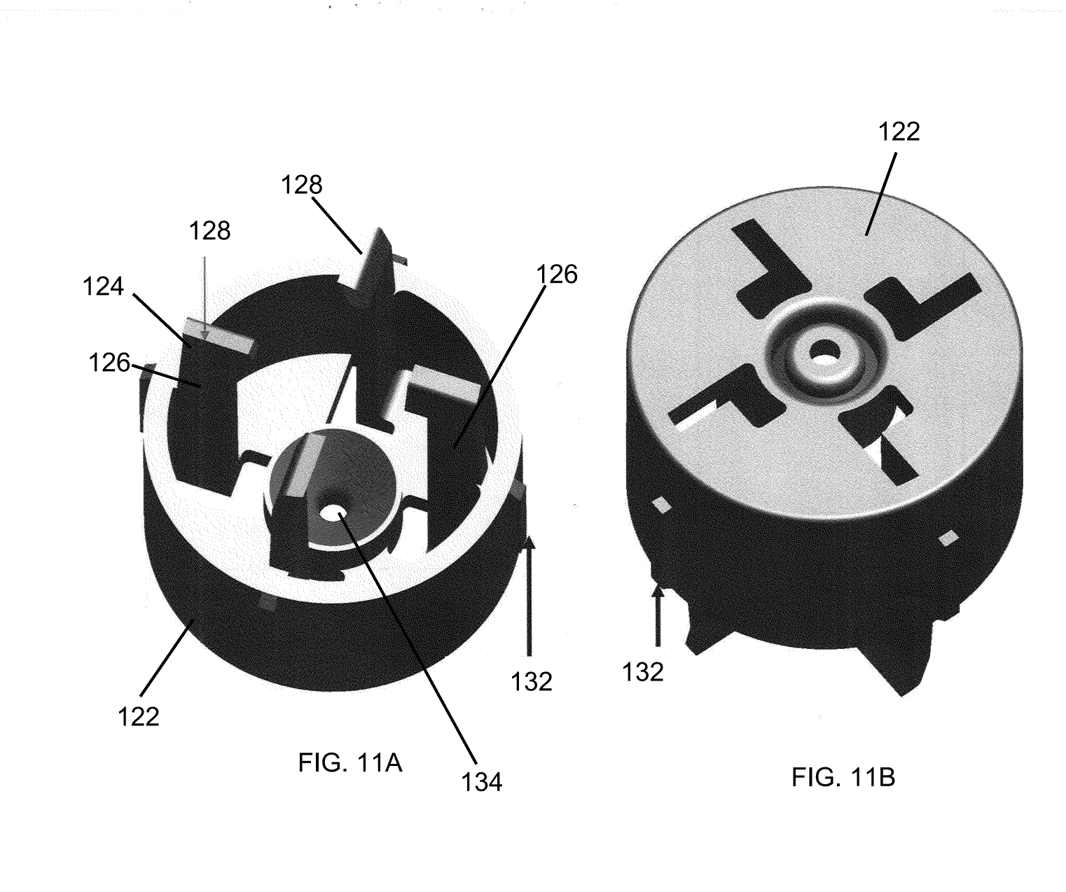

FIGS. 11A and 11B and subsequent figures illustrate a sliding shutter 122 that sliding engages the housing 102 and the needle 104. FIG. 11A a perspective view of the interior of the sliding shutter 122. As can be seen in FIG. 11A, the sliding shutter 122 includes a plurality of protrusions 124 that extend therefrom. The protrusions 124 include a first leg 126 and a second leg 128 extending inwardly toward the center of the sliding shutter 122 and substantially transverse to the first leg 126. The second legs 128 each include a hook member 130. The protrusions 124 are arranged around the periphery of the shutter 122 so as to complementarily engages the snapping cams 108 and anti-second use cams 110 during assembly and use of the needle assembly 100, as described below.

The sliding shutter 122 also includes a plurality of ribs 132 extending about the outer periphery of the sliding shutter 122. The ribs 132 are configured and arranged to align with and slidingly engage the slotted guides 106 of the housing member 102 to allow relatively sliding movement of the housing member 102 and the shutter 122, but substantially prevent relative rotation of the housing member in relation to the sliding shutter 122 and vice versa when the housing member 102 and the sliding shutter 122 are assembled together, as further described below. The sliding shutter 122 includes a central opening 134 therein configured to allow the injection member 120 to extend therethrough when the attached to the housing 102.

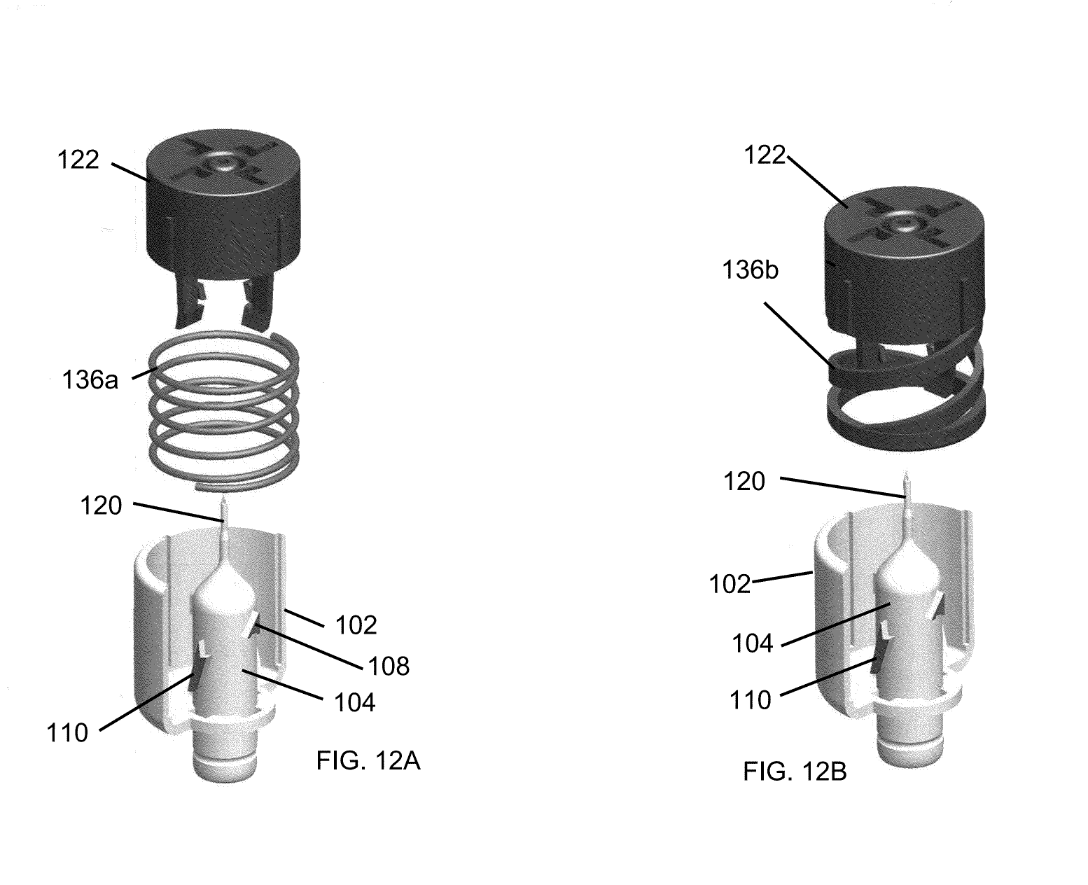

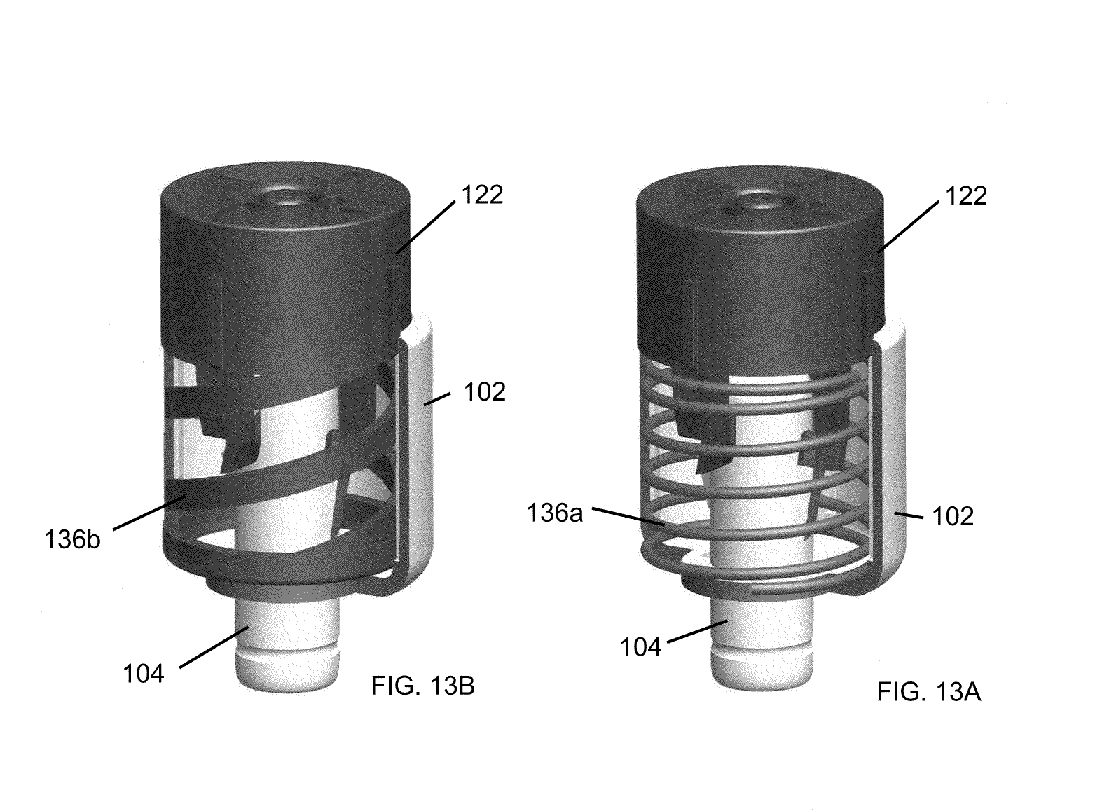

FIGS. 12A and 12B show exploded views of alternative embodiments of the device 100 that include different spring 136a, 136b configurations, whose functioning is further described below. FIGS. 13A and 13B show the different embodiments in a partially cut-away at the beginning of assembly of the housing 102 and shutter 102. In both embodiments, the springs 136a, 136b are arranged between the sliding shutter 122, needle 104 and the housing member 102. In FIG. 12A and 13A, the spring 136a is a separate component from the housing 102 and shutter 122. The spring 136a has an outer diameter that is smaller than the inside diameter of the housing 102 so that, as shown in FIG. 13A, is received within the housing 102 when assembled thereto. The outer diameter of the spring 136a is approximately the same as the outer diameter of the shutter 122 so that, when the device 100 is assembled, the upper end of the spring 136a (as oriented in FIGS. 12A and 13A), engages the lower end of the shutter. The spring 136a has sufficient length so that, upon assembly and/or operating of the assembly 100, the lower end of spring 136a engages the interior lower end of the housing 102.

In FIGS. 12B and 13B, the spring 136b, rather than being a separate component, is attached to and/or formed integral with the sliding shutter 122. The spring 136b in FIGS. 12B and 13A can be formed with the sliding shutter 122 by various methods, including molding, e.g., co-molding, over-molding, injection molding, etc., or by other attachment methods, e.g., welding, gluing, fastening, etc. or any other attachment methods that are known or may become known.

The springs 136a, 136b are formed of any material having suitable spring-like properties and strength, such as metal or plastic. In the illustrated embodiments, the springs 136a, 136b are coil springs. However, other embodiments include other types of springs, as will be appreciated by those of ordinary skill in the art.

FIG. 13A through FIG. 14B illustrates the sliding shutter 122, needle 104 and the housing member 102 being assembled. In FIGS. 14A and 14B, the springs 136a, 136b are not shown in order to more clearly show the assembly process. In FIGS. 13A and 13B, the housing 102 and shutter 122, with the spring 136a, 136b located between the housing 102 and the shutter 122, are oriented so that that ribs 132 and slots 106 align. The housing 102 and shutter 122 are moved toward each other into engagement so that the ribs 132 engage and slide into the slots 106. As described above, each slot 106 and its respective rib 132 define a key-slot locking mechanism, such that, once the ribs 132 engage their respective slots 106 the housing 102 and the shutter 122 are substantially prevented from relative rotation. However, the ribs 132 slide along the slots 106, permitting a relative sliding axial movement between the housing 102 and the shutter.

As shown in FIGS. 14A and 14B, during assembly, the sliding shutter 122 and the housing 102 are pushed together such that the shutter 122 is slide into the housing 102. As the spring 136a, 136b is located between and engages the housing 102 and the shutter 122, the spring is compressed by this movement. Accordingly, the sliding engagement of the housing 102 and the shutter 122 is against the compressive force of the spring 136a, 136b, and the spring 136a, 136b asserts an opposing biasing force against the housing 102 and the shutter 122. That is, the spring 136a, 136b biases the housing 102 and the shutter 122 in a direction apart or disengaging from each other.

As seen in FIGS. 14A and 14B, as the housing 102 and shutter 122 are slidingly engaged toward each other, two opposing protrusions 124, and more specifically, the hook portions 130 thereof, engage the sides of respective triangular snapping cams 108. These sides, being oriented at an angle to the axial movement of the shutter 122 relative to the housing 102, implement a ramping or wedging effect against the hook portion 130. This causes the protrusion 124 to be deflected sideways, in an increasing amount as the hook 130 slides downwardly along the side of the snapping cam 108. The protrusions 124 have a spring-like resiliency to them, such that a restoring force is stored in the protrusion 124 as it moves along the side of the snapping cam. When the shutter 122 and housing are sufficiently engaged with each other, e.g., pushed together, as seen in FIG. 14B, the hook portion 130 is moved down past the snapping cam 108 until it is no longer engaged with the side of the snapping cam 108. At this location, the restoring spring force moves the deflected protrusion 124 back its original undeflected position. The hook portion 130 is thus moved underneath the bottom of the snapping cam 108 and into axial alignment with the snapping cam recesses 112. The upward (in the orientation in FIG. 14B) biasing force of the spring 136a, 136b moves the shutter 122 upwardly relative to the housing such that the hook members 130 of the sliding shutter 122 engage and extend into the snapping cam 108 recesses 112. The engagement of the hook members 103 with the snapping cams prevents further upward movement of the shutter 122 relative to the housing 102. This is shown in FIG. 14B. As both ribs 132/slots 106 and the sides of the recesses 112 substantially prevent relative rotation of the housing 102 and shutter 122, the hooks 130 cannot be rotated out of substantial axial alignment with the recesses 112. Therefore, the shutter 122 cannot be moved relatively upwardly past the recesses 112, preventing disengagement of the housing 102 and shutter 122, even under the biasing force of the spring 136a, 136b. The relative upward biasing force of the spring 136a, 136b assists in keeping the hooks 130 engaged in the recesses 112.

As seen in FIG. 14B, for example, when the assembly 100 is in the assembled state, the shutter 122 fully covers or extends over the injection member 120. In this state, the tip of the injection member 120 is not exposed. This helps keep contaminants off of the injection member 120, and in the case where the injection member 120 is sharp, e.g., an needle, it reduces the risk of an accidental needle stick or injury.

FIGS. 15 through 16H show various stages of actuation and retraction of the sliding shutter 122. Similarly to needle assembly 10, the outer surface of the shutter 122, i.e., the “top” surface as seen in the figures, is placed against the skin 68 of patient. To penetrate the skin 68 with the injection member, the housing 102 and needle 104 are pressed into further engagement with the shutter 122 such that, relatively, the shutter is slidingly moved further into the housing 122, or alternatively, the injection member 120 is outwardly past the end of the shutter. The injection member 120 thus penetrates the skin 68, whereupon a substance can be injected or delivered into the skin or subdermally.

As shown in FIGS. 15 through 16D, upon the above-described actuation of the assembly 100, opposing sets legs 126, 128 of the sliding shutter 122, and more particularly the hook portions 130 thereof, begin to slide downwardly along the sloping first side surface 114 of the anti-second use cams 110. FIGS. 16A through 16D show various stages of travel by the sliding shutter 122 along the anti-second use cams 110 during actuation, e.g., depression of the sliding shutter 122 relative to the housing. As can be seen in the illustrated embodiment, because the first side surface 114 extends at an angle relative to the direction of relative movement of the shutter 122 and housing 102, and thus the hook portion 130, the first side surface 114 acts as a ramp or wedge against the hook portion 130. This causes the protrusion 124 to be deflected sideways, in an increasing amount as the hook 130 slides downwardly along the first side surface 114 of the anti-second use cam 110. This generates a restoring force in the spring-like protrusion 124 as it moves along the first side surface 114 of the anti-second use cam 110.

FIG. 16E shows the shutter 122 in a fully-depressed position, i.e., the full extension of the injection member 120 out of the shutter 122. An enlarged view of the exposed injection member 120 extending through the opening 134 in the shutter is shown in FIG. 16I. At the fully-depressed position, the hook portion 130 is moved down past the lower end of the anti-second use cam 110 until it is no longer engaged with the first side surface 114. The restoring spring force moves the deflected protrusion 124 back its original undeflected position. The hook portion 130 is thus moved underneath the bottom of the anti-second use cam 110, and as seen in FIG. 16E, on the other side of the anti-second use cam 110 adjacent to the second side surface 116.

FIGS. 16F and 16G show various stages of retraction of injection member 120 into the sliding shutter 122, or conversely, the extension of the shutter 122 back over the injection member 120. As will be understood by those of ordinary skill in the art, when the assembly is in the position shown in FIG. 16E, the spring 136a, 136b is compressed between the housing 102 and the shutter 122. When the needle assembly 100 is removed from the skin 68, e.g., after the injection procedure is complete, the force compressing the housing 102 and shutter 122 together is released. Consequently, the biasing force in the compressed spring 136a, 136b biases the housing 102 and the shutter 122 apart, causing the injection member 120 to retract relative to the shutter 122, or alternatively, causing the shutter 122 to extend back over the injection matter 120. Thus, in the sequence show in in FIGS. 16F through 16H, the shutter 122 moves upward relative to the housing to cover the exposed injection member 120 as seen in FIG. 16H.

As can be seen in FIGS. 16F and 16G, during the relative movement of the housing 102 and the shutter 122, the second legs 128 of the of the protrusions 124 contact and slide along the second sloping side 116 surface of the anti-second use cams 110. As the second side surface 114 extends at an angle relative to the direction of relative movement of the shutter 122 and housing 102, the second side surface 116 acts as a ramp or wedge against the second leg 128. This causes the second leg 128 to be deflected sideways, in an increasing amount as the second leg 128 slides upwardly along the second side surface 116 of the anti-second use cam 110 under the upward biasing force of the spring 136a, 136b. This generates a restoring force in the spring-like second leg 128 as it moves along the second side surface 116 of the anti-second use cam 110.

FIG. 16H shows the sliding shutter in an extended position where the injection member 120 no longer protrudes through the opening 134 in the sliding shutter 122. When the upward (in the orientation in the figures biasing force of the spring 136a, 136b moves the shutter 122 upwardly relative to the housing such that the hook members 130 of the sliding shutter 122 move above the top surface of the anti-second use cam 110, the hook member 130 no longer engages the second side surface 116, and the restoring force in the second legs 128 restores the second legs 128 into their undeflected position. As seen in FIG. 16H, in the undeflected position, the hooks 130 are axially aligned with the stop region 118. This prevents the sliding shutter 122 from being actuated again, i.e., from being depressed into or relative to the housing 102, or the housing 102 being advanced relative to the shutter 122 to expose the needle. More specifically, upon an attempted relative movement of the housing 102 and the shutter 122, such as when the shutter 122 is placed against the skin 68 and the needle 104 is pressed toward the skin 68, the hooks 130 are moved into engagement against the stop regions 118 and further movement in that direction is prevented. Accordingly, the shutter 122 and housing 102 cannot be moved so as to expose the injection member 120.

Moreover, the anti-rotational engagement of the ribs 132 and slots 106, along with recessed configuration of the stop region 118, substantially prevent relative rotation of the housing 102 and shutter 122. Thus, the hooks 130 cannot be rotated out of substantial axial alignment with the stop regions 118. Yet further, as described above, the engagement of the hooks 130 with the recesses 112 prevents disengagement of the housing 102 and shutter 122.

Therefore, after a single usage of the needle assembly 100, the injection member 120 is locked within the sliding shutter 122 and cannot be extended for reuse. This helps prevent accidental needle sticks after usage, especially for a medical practitioner administering the injection, lessening the chance that any microorganisms transferred from the patient to the injection member 120 during the injection will be transferred to the practitioner. It also prevents a used, potentially contaminated needle from being re-used, either accidentally or purposefully, reducing the risk of patient-to-patient transfer of disease.

As may be recognized by those of ordinary skill in the pertinent art based on the teachings herein, numerous changes and modifications may be made to the above-described and other embodiments without departing from its scope as defined in the claims. For example, the components of the needle assembly may be made of any of numerous different materials that are currently known, or that later become known for performing the function(s) of each such component. Similarly, the components of the needle assembly may take any of numerous different shapes and/or configurations. Also, the needle assembly may be used to inject any of numerous different types of fluids or other substances for any of numerous different applications, including, for example, medicaments, pharmaceuticals, vaccines, liquid nutrition products, supplements, and numerous other products that are currently known, or later become known. In addition, the characteristics of the needle assembly may be adjusted, including for example the shape and/or configuration of the flexible shield and/or the needle, to meet the requirements of any of numerous different applications and/or products to be dispensed. Accordingly, this detailed description of embodiments is to be taken in an illustrative, as opposed to a limiting sense.

What is claimed is:

1. A device, comprising:
a flexible shield movable between (i) a first position, wherein the flexible shield is uncompressed, and (ii) a second position, wherein the flexible shield is compressed; and
an administering member;
wherein at least one of the administering member and the flexible shield is axially movable with respect to the other of the administering member and the flexible shield in an unlocked position prior to and during usage of the needle assembly, and the at least one of the administering member and the flexible shield is no longer axially movable with respect to the other of the administering member and the flexible shield in a locked position.

2. A device as defined in claim 1, further comprising a removable cap and a removable seal.

3. A device as defined in claim 2, wherein the flexible shield and the administering member are sealed within the removable cap and removable seal prior to usage of the device.

4. A device as defined in claim 1, wherein the administering member is a needle.

5. A device as defines in claim 4, wherein the needle defines a tip at a distal end thereof.

6. A device as defined in claim 1, wherein the flexible shield includes a substantially cylindrical top portion mounted atop a substantially dome-shaped base portion defining an integral spring.

7. A device as defined in claim 1, wherein the flexible shield is a bellows shaped member.

8. A device as defined in claim 1, wherein the administering member is in the unlocked position prior to and during a first use of the device.

9. A device as defined in claim 1, wherein the administering member is in the locked position after a single use of the device.

10. A device as defined in claim 1, wherein the administering member is completely enclosed within the flexible shield in the locked position.

11. A device as defined in claim 1, wherein the flexible shield defines an opening at a distal end thereof extending proximally into the flexible shield, configured to align the administering member therein and prevent deflection of a distal end of the administering member.

12. A device, comprising:
a first means for shielding a second means, movable between (i) a first position, wherein the first means is uncompressed, and (ii) a second position, wherein the first means is compressed; and
a second means for administering a substance;
wherein at least one of the second means and the first means is axially movable with respect to the other of the second means and the first means in an unlocked position, and the at least one of the second means and the first means is no longer axially movable with respect to the other of the second means and the first means in a locked position.

13. A device as defined in claim 12, wherein the first means is a flexible shield and the second means is an administering member.

14. A method, comprising the comprising the following steps:
attaching a distal end of a syringe to a needle assembly comprising:
a flexible shield movable between (i) a first position, wherein the flexible shield is uncompressed, and (ii) a second position, wherein the flexible shield is compressed; and an administering member; wherein at least one of the administering member and the flexible shield is axially movable with respect to the other of the administering member and the flexible shield in an unlocked position prior to and during usage of the needle assembly, and the at least one of the administering member and the flexible shield is no longer axially movable with respect to the other of the administering member and the flexible shield in a locked position;
placing a distal end of the flexible shield adjacent a skin surface of a patient;
depressing the syringe and thereby axially moving the administering member relative to the flexible shield toward the skin surface;
moving the flexible shield from the first position to the second position;
contacting the skin surface with the administering member;
penetrating the skin surface with the administering member;
introducing a substance from the syringe through the administering member, and into the skin;
retracting the syringe and thereby withdrawing the administering member from the skin;
moving the flexible shield from the second position to the first position;
and locking the administering member within the shield.

15. A method as defined in claim 14, wherein the distal end of the syringe comprises a connector and an annular protuberance laterally extending therefrom, and the administering member comprises a corresponding annular recess, and wherein the attaching step comprises abutting a distal end of the administering member against the annular protuberance of the of the syringe connector.

16. A method as defined in claim 15, wherein the contacting step includes snapping the annular protuberance of the syringe connector into the annular recess of the administering member.

17. A method as defined in claim 14, wherein the penetrating step further comprises penetrating the skin surface with the administering member without exposing the administering member.

18. A method as defined in claim 14, wherein the retracting step further comprises retracting the syringe and thereby withdrawing the administering member from the skin without exposing the administering member.

19. A method as defined in claim 16, further comprising the step of detaching the needle assembly from the syringe.

20. A method as defined in claim 19, wherein the detaching step further comprises unsnapping the administering member from the syringe connector.
